(19) United States
(12) Patent Application Publication
(10) Pub. No.: US 2020/0146973 A1
(43) Pub. Date: May 14, 2020

(54) Pharmaceutical Composition for Oral Delivery of Hydrophobic Small Molecule Drug and Hydrophilic Small Molecule Drug Concurrently

(71) Applicant: National Tsing Hua University, Hsinchu (TW)

(72) Inventors: Hsing-Wen SUNG, Hsinchu (TW); Po-Yen LIN, Hsinchu (TW); Kuan-Hung CHEN, Hsinchu (TW)

(21) Appl. No.: 16/705,893

(22) Filed: Dec. 6, 2019

Related U.S. Application Data
(63) Continuation-in-part of application No. 16/178,041, filed on Nov. 1, 2018, now abandoned.
Application No. 16/178,041 is a continuation-in-part of application No. 15/797,413, filed on Oct. 30, 2017, now abandoned.

(30) Foreign Application Priority Data
Nov. 18, 2016 (TW) 105137833

(51) Int. Cl. A61K 9/46; A61K 9/00; A61K 47/12; A61K 47/02; A61K 31/337; A61K 31/7068; A61K 9/48

(52) U.S. Cl. CPC A61K 9/0007; A61K 9/0053; A61K 47/12; A61K 9/4891; A61K 31/337; A61K 31/7068; A61K 47/02

(57) ABSTRACT

A pharmaceutical composition for oral delivery of hydrophobic small molecule drug and hydrophilic small molecule drug concurrently is provided. The pharmaceutical composition includes an enteric layer and a drug layer, in which the drug layer is encapsulated in the enteric layer. The drug layer includes a therapeutically effective amount of a hydrophobic small molecule drug, a therapeutically effective amount of a hydrophilic small molecule drug, a lipophilic solvent, an acidic compound and an effervescent ingredient.

### RELATED APPLICATIONS

This application is a continuation-in-part of U.S. patent application Ser. No. 16/178,041, filed Nov. 1, 2018, which is a continuation-in-part of the application Ser. No. 15/797,413, filed Oct. 30, 2017, and claims priority to Taiwan Application Serial Number 105137833, filed Nov. 18, 2016, all of which are herein incorporated by reference.

### BACKGROUND

### Technical Field

The present disclosure relates to a pharmaceutical composition for oral delivery. More particularly, the present disclosure relates to a pharmaceutical composition for oral delivery of hydrophobic small molecule drug and hydrophilic small molecule drug concurrently.

### Description of Related Art

Oral administration is a convenient and user-friendly mode of drug administration, either in the form of a solid or a liquid suspension, which continues to dominate the area of drug delivery technologies. Even though many types of drugs could be administered orally with acceptable efficacy, there remains a problem for some classes of drugs, especially those which are known to have good solubility, but are extensively metabolized in the liver, easily pumped out by the intestinal epithelium (poor permeability) or irritative to the gastric mucosa. For these drugs, injection administration becomes the major option to achieve acceptable drug absorption and bioavailability which however leads to increased risk and expenses and further is painful for patients.

In addition, many common hydrophobic drugs, such as curcumin, paclitaxel and doxorubicin, have been proved to have a good therapeutic effect in experiments. However, the hydrophobicity thereof hinders them from mixing homogeneously in fabrication, or makes them hard to disperse while they disintegrate in the digestive organs, or causes them to deposit. Thus, the hydrophobic drugs are hard to be absorbed by living bodies and suffer low bioavailability. The abovementioned problems may affect the therapeutic effect, generate some side-effects, retard extensive clinical application, and impede further development of the hydrophobic drugs. Therefore, hydrophobic drugs are normally administrated in intravenous infusion. In order to avoid the inconvenience of invasive treatment, the current tendency is to develop appropriate carriers for fabricating oral hydrophobic drugs.

The common carriers for oral drugs include liposomes, nanoparticle carriers made of chitosan and γ-polyglutamic acid (γ-PGA), etc. The chitosan and γ-PGA carrier system is characterized in good gastric acid tolerance and dissolvable in the small intestine to release active ingredients. However, the fabrication process of the drugs using the chitosan and γ-PGA carrier system is very complicated and unfavorable for mass production, wherein the ingredients of the drug are mixed and dried in a special process and then enveloped in gelatin capsules. The dissolution of a capsule in the small intestine is usually incomplete and hard to control, which is likely to degrade the effect of drugs. Therefore, an improved carrier of oral hydrophobic drugs should favor the users thereof.

Therefore, there is still a need to develop a pharmaceutical composition for oral delivery of hydrophobic small molecule drug and hydrophilic small molecule drug concurrently, especially an oral self-emulsifying pharmaceutical composition with good bioavailability and stability.

### SUMMARY

According to one aspect of the present disclosure, a pharmaceutical composition for oral delivery of hydrophobic small molecule drug and hydrophilic small molecule drug concurrently is provided. The pharmaceutical composition includes an enteric layer and a drug layer, wherein the drug layer is encapsulated in the enteric layer. The drug layer includes a therapeutically effective amount of a hydrophobic small molecule drug, a therapeutically effective amount of a hydrophilic small molecule drug, a lipophilic solvent, an acidic compound and an effervescent ingredient. A molar mass of the hydrophobic small molecule drug is less than 1000 g/mol, and a molar mass of the hydrophilic small molecule drug is less than 1000 g/mol. The lipophilic solvent is for dissolving the hydrophobic small molecule drug. The effervescent ingredient generates carbon dioxide bubbles when the acidic compound is dissolved in intestinal fluid to form an acidic environment. Lipophilic tails of bile salts carry the hydrophobic small molecule drug dissolved in the lipophilic solvent to incorporate into a nanofilm around each of the carbon dioxide bubble to form a monolayer system. Then each of the carbon dioxide bubble expands and approaches an air-liquid interface in a lumen, the monolayer system transforms into a double-layer nano-assembly having an inner layer and an outer layer, the hydrophilic small molecule drug is embedded in a gap formed between the inner layer and the outer layer of the double-layer nano-assembly, and lipid oil drops containing the hydrophobic small molecule drug are formed when the carbon dioxide bubbles burst at the air-liquid interface in the lumen.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1 is an ultrasonic image showing an interface of liquid and air according to the present disclosure.

FIG. 2A is a fluorescent image captured by a confocal microscope to show bubble carriers in water according to the present disclosure.

FIG. 2B is a diagram schematically illustrating a monolayer of the solvent molecules, poorly water-soluble drug and carbon dioxide bubbles according to the present disclosure.

FIG. 3A is a fluorescent image captured by a confocal microscope to show bubble carriers on water according to the present disclosure.

FIG. 3B is a diagram schematically illustrating double-layer nano-assemblies of solvent molecules, poorly water-soluble drug and carbon dioxide bubbles according to the present disclosure.

FIG. 4 shows the results of the drug release experiments of a poorly water-soluble drug in different dosage forms.

FIG. 5 shows the distributions of poorly water-soluble drug in different dosage forms in tissues of living bodies in different groups.

FIGS. 6A, 6B, 6C, 6D and 6E are ultrasonic images of carbon dioxide bubbles obtained under different conditions.

FIG. 7 shows average sizes of the lipid oil drop under different conditions.

FIG. 8 is a schematic diagram illustrating mechanism of formation of a monolayer system, a double-layer nano-assembly and lipid oil drops according to one embodiment of the present disclosure.

FIG. 9 shows analysis results of the fluorescence microscopy and TEM.

FIG. 10A is a schematic diagram of the lipid oil drops according to the present disclosure.

FIGS. 10B, 100 and 10D show structural analysis results of the lipid oil drops according to one example of the present disclosure.

FIG. 11 shows analysis results of in vivo transport route of the pharmaceutical composition of the present disclosure.

FIG. 12 shows analysis results of the immunofluorescence staining of RAW 264.7 cells.

FIG. 13 shows analysis results of the immunofluorescence staining of the MLNs.

FIG. 14 shows biodistribution of fluorescent model drugs in test rats with different administrations.

FIGS. 15A and 15B show pharmacokinetics of the pharmaceutical composition of the present disclosure.

FIG. 16 shows analysis result of hematoxylin and eosin staining.

FIGS. 17A and 17B show analysis results of the dose dependent study of the pharmaceutical composition of the present disclosure.

FIGS. 18A, 18B, 19 and 20 show analysis results of the therapeutic efficacy of the pharmaceutical composition of the present disclosure.

FIG. 21 shows body weight curve of the tumor rats after different administrations.

### DETAILED DESCRIPTION

The present disclosure will be described in detail with embodiments and attached drawings below. However, these embodiments are only to exemplify the present disclosure but not to limit the scope of the present disclosure. In addition to the embodiments described in the specification, the present disclosure also applies to other embodiments. Further, any modification, variation, or substitution, which can be easily made by the persons skilled in that art according to the embodiment of the present disclosure, is to be also included within the scope of the present disclosure, which is based on the claims stated below. Although many special details are provided herein to make the readers more fully understand the present disclosure, the present disclosure can still be practiced under a condition that these special details are partially or completely omitted. Besides, the elements or steps, which are well known by the persons skilled in the art, are not described herein lest the present disclosure be limited unnecessarily. Similar or identical elements are denoted with similar or identical symbols in the drawings. It should be noted: the drawings are only to depict the present disclosure schematically but not to show the real dimensions or quantities of the present disclosure. Besides, matterless details are not necessarily depicted in the drawings to achieve conciseness of the drawings.

### 1st Embodiment

A pharmaceutical composition is provided to form self-emulsified lipid oil drops as bubble-carrier for oral delivery, which is a mixture of a poorly water-soluble drug, a lipophilic or amphiphilic solvent, an acid initiator and a foaming agent. Next, the pharmaceutical composition may be in a gelatin capsule that is then coated with an enteric polymer. The solvent may include lipophilic fatty acids, phospholipid, triglyceride, lipid derivatives, or ester derivatives, and in one embodiment, the solvent is capric acid. The foaming agent may include carbonates or bicarbonates. The acid initiator may include organic acids or organic anhydrides. The acid initiator may be selected from a group including tartaric acid, malic acid, maleic acid, fumaric acid, succinic acid, lactic acid, ascorbic acid, amino acid, glycolic acid, adipic acid, citric acid, diethylenetriaminepentaacetic dianhydride (DTPA anhydride), citric acid anhydride, succinic acid anhydride, and combinations thereof. In one embodiment, the acid initiator is critic acid. The foaming agent, for example but not limited, is sodium bicarbonate. It is noted that citric acid and sodium bicarbonate may rapidly react with each other in water to produce carbon dioxide of gas bubbles that are present in soda at a high pressure. Furthermore, capric acid is a lipid-based fatty acid oil to be deprotonated upon exposure to water and acts as a solvent for poorly water-soluble drug. In one example of the present disclosure, the pharmaceutical composition includes various weights as follows: the poorly water-soluble drug of paclitaxel of 1-3 (±30%) mg; the solvent of capric acid of 15-60 (±10%) mg; the acid initiator of citric acid of 2-25 (±15%) mg and the foaming agent of sodium bicarbonate of 1-20 (±15%) mg.

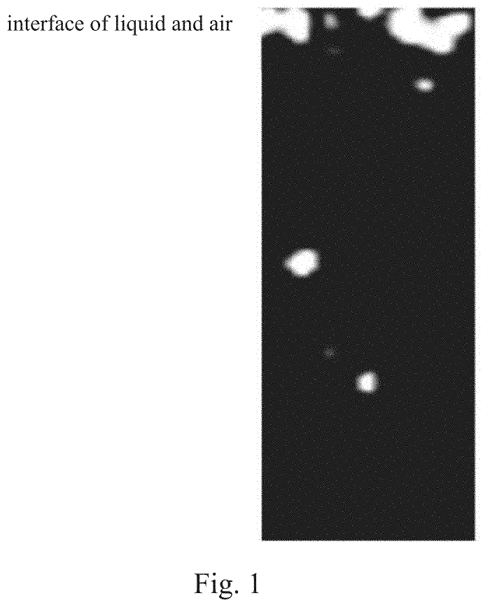

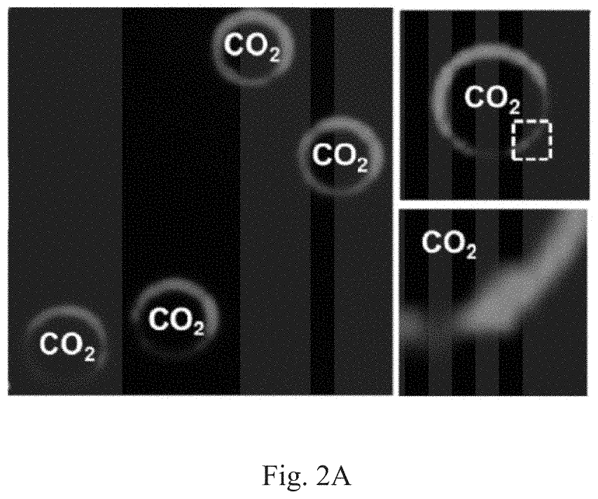

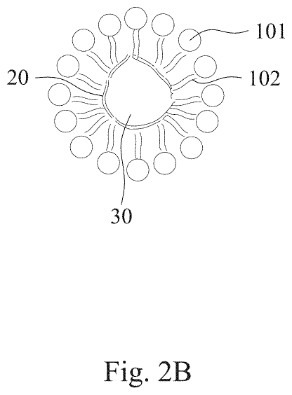

The pharmaceutical composition of the enteric-coated capsule of the present disclosure performs oral administration and dissolution in small intestine of a living body that is also an aqueous environment. While the pharmaceutical composition is exposed to the aqueous environment in an intestinal tract, the acid initiator is dissolved in the intestinal fluid to form an acidic environment in which the foaming agent of sodium bicarbonate decomposes to produce carbon dioxide bubbles. An interface of liquid and air may be seen by an ultrasonic image like one in FIG. 1. Next, please refer to FIG. 2A and FIG. 2B, these carbon dioxide bubbles 30 may be surrounded, and so stabilized by a monolayer of the amphiphilic bile salts with the nanofilm of solvent molecules (capric acid) dissolving the pharmaceutical composition anchored to the hydrophilic ends 101 of the amphiphilic bile salts. It is noted that the bile salts are derived from in small intestine of a living body, such as intrinsic amphiphilic bile salts or their derivatives. Shown in FIG. 2A and FIG. 2B, the lipophilic ends 102 of the bile salts surround one carbon dioxide bubble 30, and the nanofilm 20 that includes the poorly water-soluble drug dissolved in the solvent is anchored to form the self-assembled monolayer carrier system.

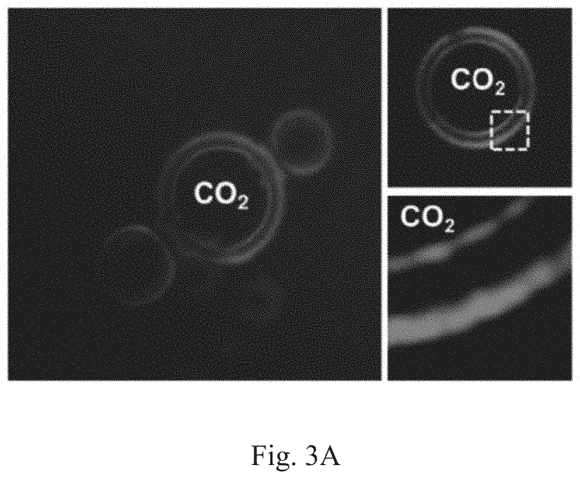

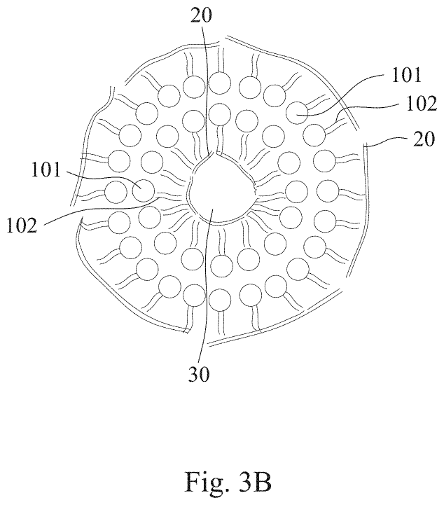

Next, the carbon dioxide bubbles 30 expand, rise and approach the interface of intestinal lumen, the self-assembled monolayer carrier system is transformed into double-layer nano-assemblies like ones in FIG. 3A and FIG. 3B. Shown in FIG. 3A and FIG. 3B, the hydrophilic ends 101 of the bile salts and the hydrophilic ends 101 of the self-assembled monolayer carrier system move toward each other and attract mutually to form double-layer nano-assemblies. Besides, the nanofilm 20 is anchored to the lipophilic tails 102 of the bile salts that moves toward the self-assembled monolayer carrier system. After the carbon dioxide bubbles 30 of the double-layer nano-assemblies burst at the interface of liquid and lumen, the solvent molecules (capric acid) and poorly water-soluble drug such as paclitaxel or curcumin and like are converted into oil-structured nano-emulsions via self-emulsification. Such the oil-structured nano-emulsions are viewed as self-emulsified drug-loaded lipid oil drops. Furthermore, the self-emulsified drug-loaded lipid oil drops are then internalized by M cells, most of which are located in Peyer's patches, and ultimately accumulated in pancreatic tumors via intestinal lymphatic transport. Accordingly, the formation of carbon dioxide bubbles generates forces that promote the efficiency of dispersion of lipophilic solvent molecules with paclitaxel or curcumin and thus aggregation is prevented. At the bursting of the bubbles, the mechanical forces rip the double-layer nano-assemblies into oil-structured nano-emulsions. The encapsulation of paclitaxel or curcumin and like molecules in the lipid oil drops that are self-emulsified in the intestinal environment is a very important factor for their stabilization and absorption.

It is noted that the pharmaceutical composition for oral delivery that may form the self-emulsified lipid oil drops as nano-carriers of the present disclosure may be fabricated into tablets, capsules, or other oral dosage forms. Besides, the enteric coating may include a methacrylic acid copolymer, hypromellose phthalate, hydroxypropyl cellulose acetate, hydroxypropyl cellulose succinate, or carboxy methyl ethyl cellulose. While the self-emulsified lipid oil drops as nano-carriers for oral delivery is swallowed by a living body, the enteric coating can protect the pharmaceutical composition for oral delivery against the attack of gastric acid in the stomach. After entering the small intestine, the enteric coating of the pharmaceutical composition is dissolved. Moreover, pharmaceutical composition for oral delivery of the present disclosure may also include excipients, carriers, diluents, flavors, sweeteners, preservatives, antioxidants, humectants, buffer agents, release-control components, dyes, adhesives, suspending agents, dispersants, coloring agents, disintegrating agents, film forming agents, lubricants, plasticizers, edible oils, or combinations thereof.

Accordingly, the pharmaceutical composition for oral delivery that may form self-emulsified lipid oil drops as nano-carriers of the present disclosure is applied to transport a poorly water-soluble drug inside a living body. The hydrophobicity makes the poorly water-soluble drug hard to be dispersed uniformly inside a living body and thus hard to be absorbed by the living body, causing a problem of low bioavailability. In one embodiment, the poorly water-soluble drug includes curcumin, paclitaxel, doxorubicin, or another active ingredient hard to dissolve in water.

These are always the focuses of medicine research: improving low solubility, transporting instable or high-toxicity medicine, increasing the amount of the medicine transported to the target tissue, and improving the efficiency of transporting macromolecule medicine into cells. Many of anticancer drugs, anti-AIDS drugs, and immunotherapy drugs are bulky polycyclic compounds of low aqueous solubility and feature hydrophobicity. The hydrophobicity assists these drugs to pass through the lipid bilayer membrane and enter into the cells in some extent and increases the specificity of the drugs to special cell receptors. However, the application thereof usually encounters many difficulties. In oral administration, hydrophobic drugs normally have low absorptivity and poor bioavailability. In intravenous administration, hydrophobic drugs are hard to disperse and likely to block blood vessels and respiratory tracts. Besides, low dispersity also causes the drugs to condense in high concentration, which is likely to induce local toxicity in the body and hinder the drugs from entering blood circulation. Thus, the drugs are hard to absorb and low in bioavailability.

The objective of the present disclosure is to provide self-emulsified lipid oil drops as nano-carriers for oral delivery able to effectively transport poorly water-soluble drugs, whereby to overcome the problems encountered in developing hydrophobic drugs. Below, drug-release experiments and animal experiments are used to demonstrate the present disclosure. In following embodiments but not limit to, curcumin may be used to exemplify the poorly water-soluble drug and verify the bioavailability of the self-emulsified lipid oil drops as nano-carriers.

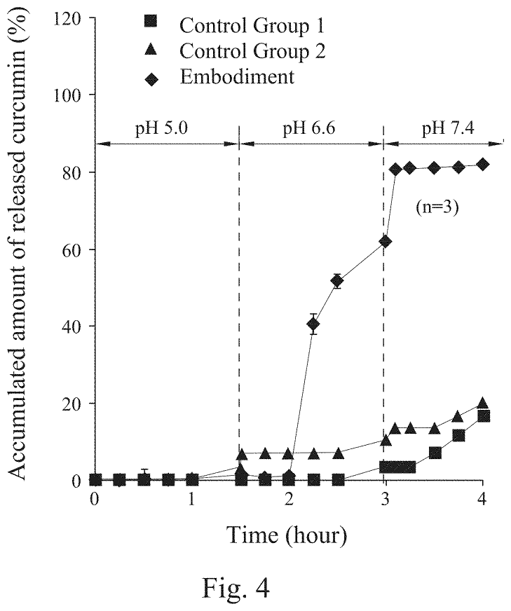

Refer to FIG. 4 for the results of in vitro drug-release experiments for different dosage forms. The embodiment group used in the experiments, but not limited to in the present, adopts the pharmaceutical composition for oral delivery containing curcumin as claimed as the present disclosure. Control Group 1 uses free-form curcumin without any additive. Control Group 2 uses free-form curcumin with sodium bicarbonate (SBC) added. The compositions of the embodiment group and the control groups are all fabricated into capsules with enteric coating. The capsules of each group are placed in a dialysis bag (MWCO 100 kDa), and the pH buffer, which simulates the physiological environment, is used as the dialysis solution. The dialysis bag is placed and persistently oscillated in an oscillation water bath at a constant temperature of 37° C. The dialysis solution is sampled at specified time points. High-performance liquid chromatography (HPLC) is used to detect the drug released by the bubble carriers in different pH environments. It is observed in FIG. 4: after the experiments have been undertaken for 2 hours, the drug release ratio of the pharmaceutical composition for oral delivery of the present disclosure is significantly higher than that of the compositions of the control groups. Therefore, the pharmaceutical composition for oral delivery of the present disclosure is proved to have very high drug release efficiency.

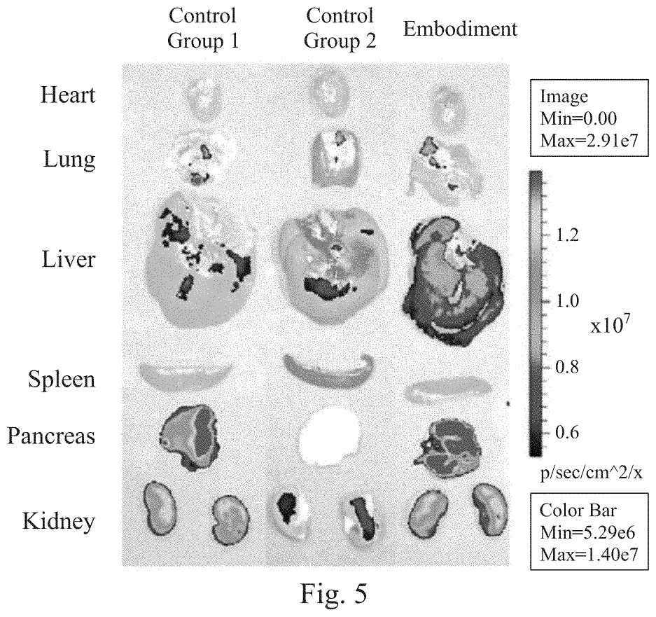

Refer to FIG. 5 showing the distribution of the hydrophobic ingredient of different dosage forms in the tissue of living bodies. Wistar rats (each weighing 300-500 g) are used in the experiments using the in-vivo imaging system (IVIS). In the embodiment of the present disclosure for the experiments, the curcumin-containing pharmaceutical composition for oral delivery of the present disclosure is orally delivered with feeding needles to the stomachs of the rats. In Control Group 1, the free-form curcumin is injected hypodermically into the rat. In Control Group 2, the free-form curcumin is orally delivered with feeding needles to the stomachs of the mice. After having taken the drugs for 2 hours, the rats are sacrificed with carbon dioxide. The fresh soft tissues of the rat, including hearts, lungs, livers, spleens, pancreases, and kidneys, are excised, washed, and placed on the imaging bed. Then, the soft tissues are imaged instantly with IVIS. The tissues and bodies of the rats are handled according to the regulations for experimental animals. The primitive data acquired with IVIS is reconstructed and analyzed with the image reconstruction and analysis software to learn the in vivo distribution of the multifunctional oral micro particles. In the experiments, the molecular imaging system of IVIS is used to assist in positioning the tissues, and the regions of interest (ROI) of the organs/tissues absorbing drugs are manually selected for quantitative analysis. Thus is acquired the absorptivity of each organ/tissue and the pharmacokinetic distribution of the curcumin-containing compositions.

Refer to FIG. 5, in comparison with Control Group 1 (injecting free-form curcumin hypodermically) and Control Group 2 (delivering free-form curcumin orally), the embodiment of the present disclosure performs higher absorptivity in livers, pancreases, and kidneys of the rats. Thus, pharmaceutical composition for oral delivery to form the self-emulsified lipid oil drops as nano-carriers of the present disclosure has good bioavailability.

In conclusion, while exposed to water, the pharmaceutical composition for oral delivery is able to form the self-emulsified lipid oil drops as nano-carriers. The pharmaceutical composition for oral delivery generates monolayer bubble structures containing poorly water-soluble drug that can be converted into double-layer bubble structures containing poorly water-soluble drug near the interface of water and lumen. While the carbon dioxide bubbles of the double-layer nano-assemblies burst at the interface, oil-structured nano-emulsions that contain paclitaxel via self-emulsification can be formed in a living body. The abovementioned bubble structures can effectively transport the poorly water-soluble drug to the recipient organs or tissues of living bodies. Further, the release efficiency of the poorly water-soluble drug of the present disclosure is higher than that of the conventional dosage form. Therefore, the present disclosure is highly bioavailable, able to break through the limitation of traditional hydrophobic drugs and provide different directions of drug development.

### 2nd Embodiment

A pharmaceutical composition for oral delivery of hydrophobic small molecule drug and hydrophilic small molecule drug concurrently is provided. The pharmaceutical composition includes an enteric layer and a drug layer, wherein the drug layer is encapsulated in the enteric layer. The drug layer includes a therapeutically effective amount of a hydrophobic small molecule drug, a therapeutically effective amount of a hydrophilic small molecule drug, a lipophilic solvent, an acidic compound and an effervescent ingredient. A molar mass of the hydrophobic small molecule drug is less than 1000 g/mol, and a molar mass of the hydrophilic small molecule drug is less than 1000 g/mol. The lipophilic solvent is for dissolving the hydrophobic small molecule drug. The effervescent ingredient generates carbon dioxide bubbles when the acidic compound is dissolved in intestinal fluid to form an acidic environment. Lipophilic tails of bile salts carry the hydrophobic small molecule drug dissolved in the lipophilic solvent to incorporate into a nanofilm around each of the carbon dioxide bubble to form a monolayer system. Then each of the carbon dioxide bubble expands and approaches an air-liquid interface in a lumen, the monolayer system transforms into a double-layer nano-assembly having an inner layer and an outer layer, the hydrophilic small molecule drug is embedded in a gap formed between the inner layer and the outer layer of the double-layer nano-assembly, and lipid oil drops containing the hydrophobic small molecule drug are formed when the carbon dioxide bubbles burst at the air-liquid interface in the lumen.

The pharmaceutical composition can further include a gelatin layer, wherein the drug layer is coated with the gelatin layer. Therefore, the drug layer of the pharmaceutical composition can be in a gelatin capsule that is then coated with the enteric layer. In addition, the pharmaceutical composition can be in form of a tablet or a capsule. The enteric layer can include a methacrylic acid copolymer, hypromellose phthalate, hydroxypropyl cellulose acetate, hydroxypropyl cellulose succinate, or carboxymethyl ethyl cellulose. The hydrophobic small molecule drug can include curcumin, paclitaxel, doxorubicin, cisplatin, mitomycin C, etoposide, irinotecan or tamoxifen, wherein the molar mass of curcumin, paclitaxel, doxorubicin, cisplatin, mitomycin C, etoposide, irinotecan and tamoxifen is 368.38 g/mol, 853.906 g/mol, 543.52 g/mol, 300.01 g/mol, 334.332 g/mol, 588.557 g/mol, 586.678 g/mol and 371.515 g/mol, respectively. The hydrophilic small molecule drug can include gemcitabine or 5-fluorouracil, wherein the molar mass of gemcitabine and 5-fluorouracil is 263.198 g/mol and 130.077 g/mol, respectively. The lipophilic solvent can be C6-C10 fatty acid. Preferably, the lipophilic solvent can be capric acid. The acidic compound can be citric acid. The effervescent ingredient can be sodium bicarbonate. It is noted that citric acid and sodium bicarbonate may rapidly react with each other in water to produce carbon dioxide of gas bubbles that are present in soda at a high pressure. In one example of the present disclosure, the lipophilic solvent, the acidic compound and the effervescent ingredient of the drug layer can be contained in a weight ratio of 18:2:1 to 18:8:7.

### 1. Optimization of Formulation and Structural Analysis of the Pharmaceutical Composition of Present Disclosure

To optimize the formulation in each pharmaceutical composition of present disclosure for forming an appropriate acidic environment to generate carbon dioxide bubbles, an enteric-coated gelatin capsule that contains a powdered mixture of gemcitabine (1 mg), paclitaxel (1 mg), capric acid (18 mg), sodium bicarbonate (5 mg) and a predetermined dose of citric acid (0, 2, 4, 6, or 8 mg). The contents of each enteric-coated gelatin capsule with various amounts of citric acid are initially exposed to deionized (DI) water. The formation of the carbon dioxide bubbles and changes in their citric acid content in DI water are then monitored using a camera and an ultrasonic instrument.

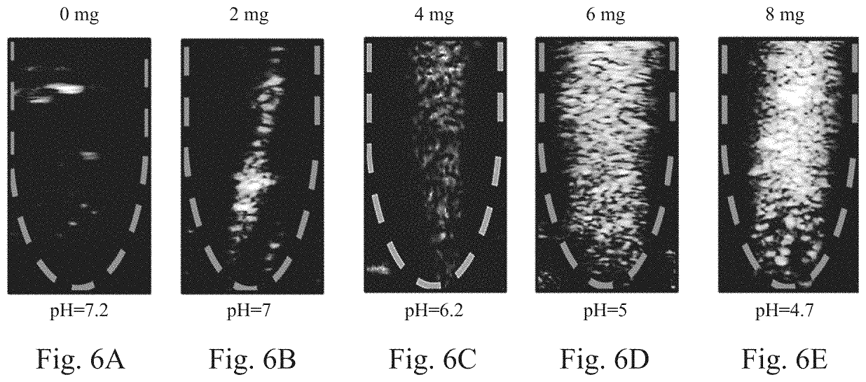

Please refer to FIGS. 6A, 6B, 6C, 6D and 6E, which are ultrasonic images of carbon dioxide bubbles obtained under different conditions. In detail, the ultrasonic image of FIG. 6A shows the carbon dioxide bubbles generating by the enteric-coated gelatin capsule contains 0 mg of citric acid, and the pH value of the solution in this test group is 7.2. FIG. 6B shows the carbon dioxide bubbles generating by the enteric-coated gelatin capsule contains 2 mg of citric acid, and the pH value of the solution in this test group is 7. FIG. 6C shows the carbon dioxide bubbles generating by the enteric-coated gelatin capsule contains 4 mg of citric acid, and the pH value of the solution in this test group is 6.2. FIG. 6D shows the carbon dioxide bubbles generating by the enteric-coated gelatin capsule contains 6 mg of citric acid, and the pH value of the solution in this test group is 5. FIG. 6E shows the carbon dioxide bubbles generating by the enteric-coated gelatin capsule contains 8 mg of citric acid, and the pH value of the solution in this test group is 4.7. The results show that the citric acid content of 6 mg is the most appropriate. Thus, the citric acid content of 6 mg is selected for further experiments.

To optimize the formulation in each pharmaceutical composition of present disclosure for generating an appropriate size of carbon dioxide bubbles, the enteric-coated gelatin capsule that contains a powdered mixture of gemcitabine (1 mg), paclitaxel (1 mg), capric acid (18 mg), citric acid (6 mg) and a predetermined dose of sodium bicarbonate (1, 3, 5, or 7 mg). The contents of each enteric-coated gelatin capsule with various amounts of sodium bicarbonate are initially exposed to deionized (DI) water. The average particle sizes of the lipid oil drops formed in their sodium bicarbonate content in DI water are analyzed by a dynamic light scattering (DLS, Zetasizer Nano-ZS, Malvern, Worcestershire, UK).

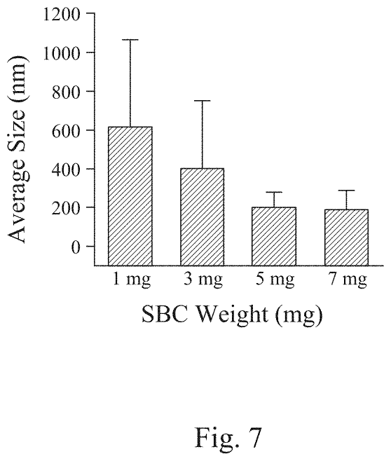

Please refer to FIG. 7, which shows average particle sizes of the lipid oil drops under different conditions. In FIG. 7, the average particle size of the lipid oil drops formed by the enteric-coated gelatin capsule containing 1 mg, 3 mg, 5 mg, and 7 mg of sodium bicarbonate is about 600 nm, 400 nm, 200 nm and 200 nm, respectively. The results show that the sodium bicarbonate content of 5 mg is the most appropriate. Thus, the sodium bicarbonate content of 5 mg is selected for further experiments.

For studying the formation of the monolayer system and their structural changes as the double-layer nano-assembly transformed into drug-laden lipid oil drops, a powdered mixture of 1 mg of Rhodamine B, 1 mg of DiO, 18 mg of capric acid, 6 mg of citric acid and 5 mg of sodium bicarbonate is placed in a confocal dish, and 1 mL of simulate intestinal fluid (pH=6.4) is added into the confocal dish, wherein Rhodamine B represents the hydrophilic small molecule drug, and DiO represents the hydrophobic small molecule drug. The process of the formation of the monolayer system and their structural changes as the double-layer nano-assembly transformed into drug-laden lipid oil drops is observed by the fluorescence microscopy, and the structural changes are analyzed by the DLS and TEM (JEOL 2010F, Tokyo, Japan).

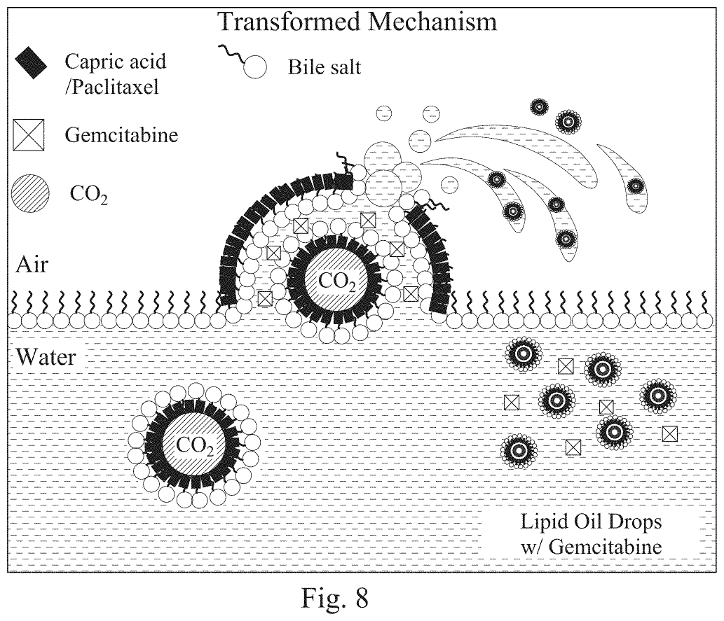

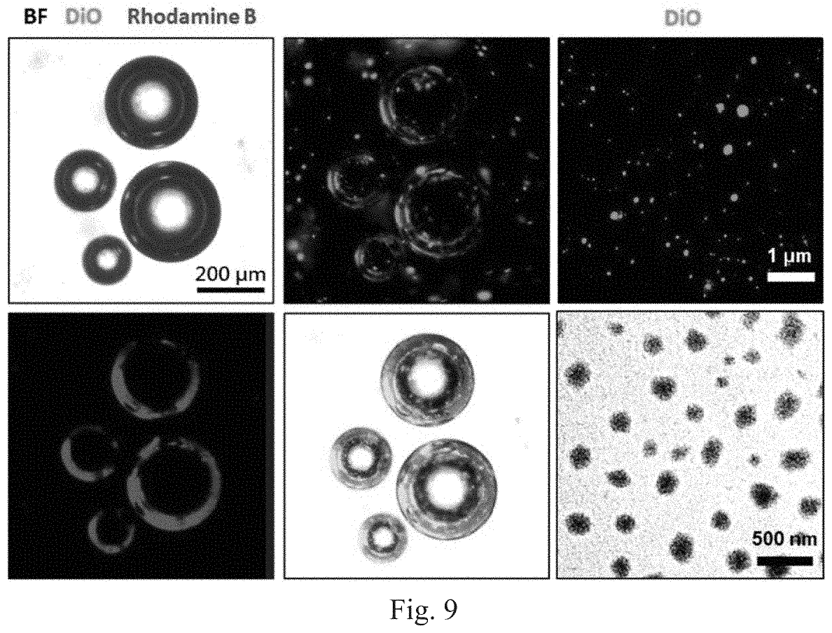

Please refer to FIGS. 8 and 9; FIG. 8 is schematic diagram illustrating mechanism of formation of a monolayer system, a double-layer nano-assembly and lipid oil drops according to one embodiment of the present disclosure, and FIG. 9 shows analysis results of the fluorescence microscopy and TEM.

In FIG. 8, following oral administration of the pharmaceutical composition of present disclosure and its dissolution in the small intestine, the citric acid is exposed to the intestinal fluid, forming an acidic environment, in which the sodium bicarbonate decomposes to generate carbon dioxide bubbles that are stabilized by a monolayer of bile salts. The lipophilic tails of these bile salts cause them to self-assemble into a nanofilm into which is incorporated the hydrophobic small molecule drug. As the carbon dioxide bubbles expand, rise, and approach the water/air interfaces in the intestinal lumen, this self-assembled monolayer system becomes double-layer nano-assemblies. After the bubbles burst in the air, the bile salts readily convert the nano-assemblies into oil-structured nano-emulsions that contain the hydrophobic small molecule drug (drug-loaded lipid oil drops) via self-emulsification. The self-emulsified drug-loaded lipid oil drops are then internalized by the enterocytes in the small intestine, ultimately accumulating in the pancreatic cancerous tissues via intestinal lymphatic transport. Lipid-based excipients are known to promote the oral absorption of drugs by increasing the fluidity of the cell membrane and lymphatic transport, resulting in enhanced bioavailability.

In FIG. 9, the red fluorescent signal (Rhodamine B) appears in the gap formed between the inner layer and the outer layer of the double-layer nano-assembly, and the green fluorescent signal (DiO) appears in the inner layer around each of the carbon dioxide bubble and the outer layer of the double-layer nano-assembly. When the carbon dioxide bubbles burst, the double-layer nano-assemblies are immediately transformed into DiO-laden lipid oil drops.

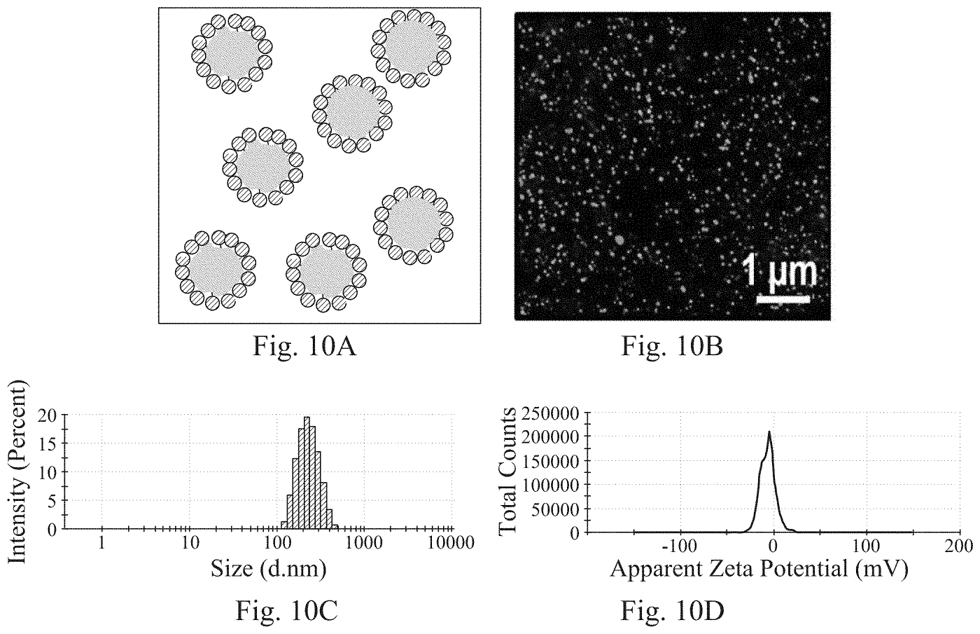

Please further refer to FIGS. 10A, 10B, 100 and 10D, FIG. 10A is a schematic diagram of the lipid oil drops according to the present disclosure, and FIGS. 10B, 100 and 10D show structural analysis results of the lipid oil drops according to one example of the present disclosure, wherein FIG. 10B is the image of the fluorescence microscopy, and FIGS. 100 and 10D are analysis results of DLS.

In FIGS. 10A and 10B, the lipophilic alkyl tails of bile salts cause them to self-assemble into lipid oil drops that incorporate DiO. The size distribution and surface charge of the DiO-laden lipid oil drops are evaluated by the DLS. According to FIGS. 100 and 10D, the average particle size of the DiO-laden lipid oil drops is about 198±66 nm, and they have a zeta potential of about −8.3±10 mV.

### 2. In Vivo Transport Route of the Pharmaceutical Composition of Present Disclosure

In addition to the comprehensive in vitro characterization of the pharmaceutical composition of the present disclosure, determination of the biodistribution of these lipid oil drops following in vivo administration is crucial because they act at the site of accumulation. To trace the pharmaceutical composition of the present disclosure in rats, DiO and Rhodamine B are used as fluorescent model drug for hydrophobic small molecule drug and hydrophilic small molecule drug, respectively.

The animal studies involved Lewis rats with masses of approximately 250 g and are performed in compliance with the “Guide for the Care and Use of Laboratory Animals”, which was prepared by the Institute of Laboratory Animal Resources, National Research Council, and published by the National Academy Press in 1996. The Institutional Animal Care and Use Committee of National Tsing Hua University approved all studies. To prepare the enteric-coated gelatin capsules for use in this experiment, hard gelatin capsules (size 9; Torpac Inc., Fairfield, N.J., U.S.A.) are manually filled with a powdered mixture of Rhodamine B (1 mg), DiO (1 mg), capric acid (18 mg), citric acid (6 mg), and sodium bicarbonate (5 mg), as per the manufacturer's instructions. In detail, the rats are oral administrated with the enteric-coated gelatin capsules that contained the pharmaceutical composition of the present disclosure (the dose of DiO and Rhodamine B is 12 mg/kg, respectively) using a loop method, which has been widely used for studying the intestinal absorption mechanisms of drugs. Furthermore, some of rats are treated with cycloheximide, which is an intestinal lymphstic inhibitor for blocking oil absorption, before the oral administrated with the enteric-coated gelatin capsules that contained the pharmaceutical composition of the present disclosure as the control group. The route of the hydrophobic small molecule drug and the hydrophilic small molecule drug delivery is then investigated by immunofluorescence staining. In detail, the animals are sacrificed 6 hours after oral administration. The small intestine, mesenteric lymph nodes (MLNs), and tumor are retrieved from euthanized rats, and the tissues are fixed in formalin for 4 hours at room temperature. Then the tissue was cut to size and embedded in OCT gel for frozen section and subsequent staining. The sections are finally observed with a confocal laser scanning microscopy (CLSM).

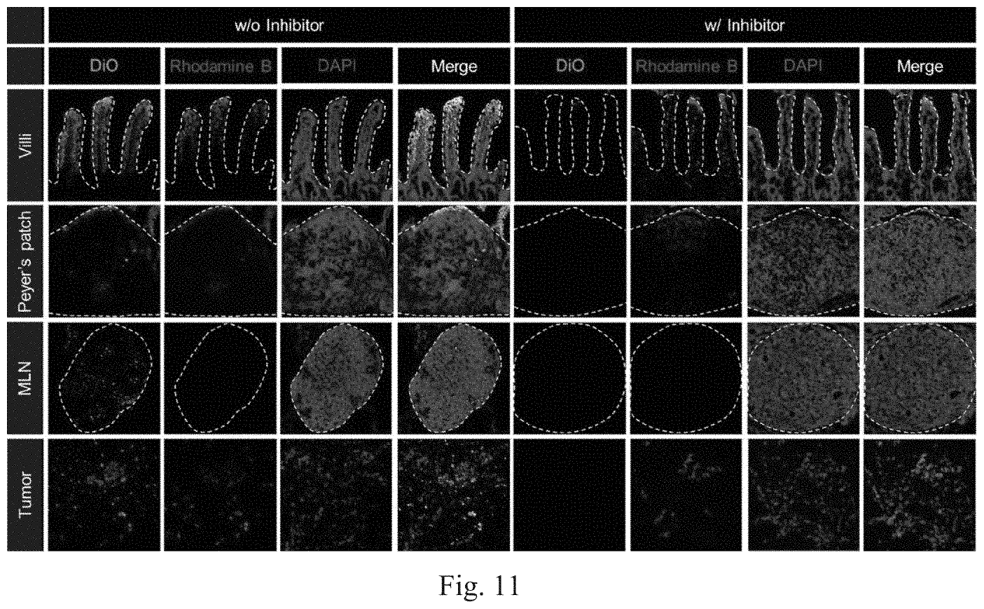

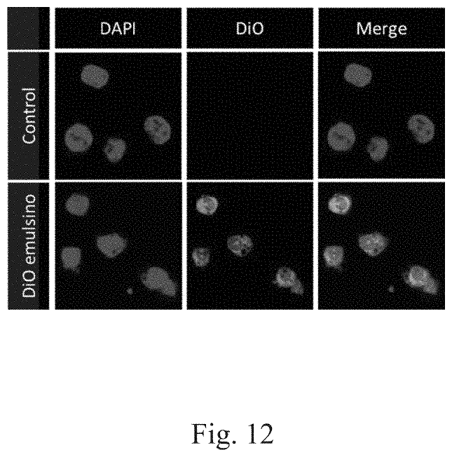

Please refer to FIG. 11, which shows analysis results of in vivo transport route of the pharmaceutical composition of the present disclosure. In FIG. 11, analysis results of the immunofluorescence staining demonstrate that the signals of DiO-laden lipid oil drops are located on the lateral surfaces of the villi. Following uptake by the M cells, the DiO-laden particles are detected in Peyer's patches and ultimately accumulated in MLNs and tumor. In contrast, no signals of DiO-laden lipid oil drops can be detected in villi, Peyer's patches, MLNs and tumor in the control group. In addition, analysis results of the immunofluorescence staining demonstrate that the signals of Rhodamine B are located on the lateral surfaces of the villi, Peyer's patches and tumor. The results indicate that the hydrophobic small molecule drug is delivered by the mesenteric transport and the hydrophilic small molecule drug is delivered by blood circulation.

To understand better the route of drug delivery, RAW 264.7 cells are added the prepared emulsion containing DiO and Rhodamine B and then cultured for 3 hours. The unabsorbed emulsion is washed with PBS, and then the cells are fixed with formalin, stained, and observed with the CLSM. In addition, the rats are oral administrated with the enteric-coated gelatin capsules that contained the pharmaceutical composition of the present disclosure (the dose of DiO and Rhodamine B is 12 mg/kg, respectively). The animals are sacrificed 6 hours after oral administration, the MLNs are retrieved from euthanized rats, and the tissues are fixed in formalin for 4 hours at room temperature. Then the tissue was cut to size and embedded in OCT gel for frozen section and subsequent staining. The sections are finally examined by CLSM.

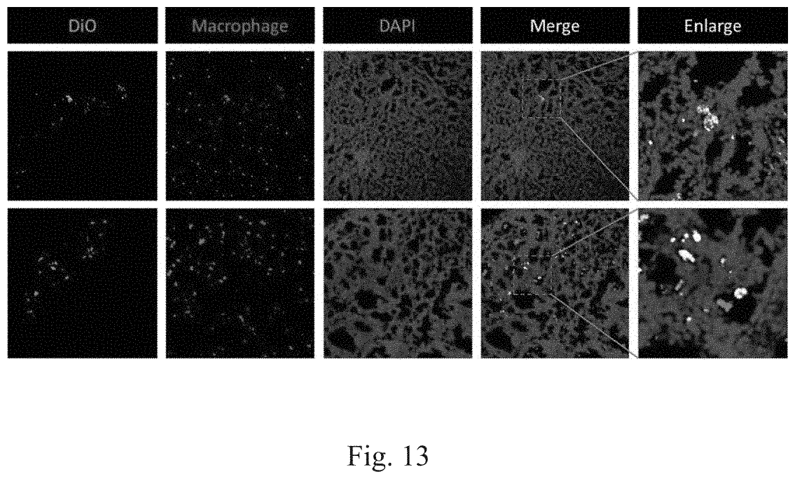

Please refer to FIGS. 12 and 13, FIG. 12 shows analysis results of the immunofluorescence staining of RAW 264.7 cells, and FIG. 13 shows analysis results of the immunofluorescence staining of the MLNs. In FIG. 12, at 3 hours of incubation, the intracellular colocalization of green fluorescence (DiO) and blue fluorescence (DAPI) is clearly visible, indicating that the DiO can be uptake into RAW 264.7 cells. In FIG. 13, the signals of DiO-laden lipid oil drops are highly colocalized with macrophages. The results indicate that the intestinal lymphatic system provides a unique route for the hydrophobic small molecule drug delivery, with the potential of passive targeting of the pancreas by mesenteric transport. Thus, the pharmaceutical composition of the present disclosure can be effectively transepithelial transport in a membrane phagocytic mode to break through the mucosal barrier, enter the macrophage by endocytosis, and accumulate in the mesenteric lymph nodes and tumor.

Further, ex vivo imaging of the distributions of different drugs in the major organs that are isolated from the test rats was conducted using an in vivo imaging system (IVIS). The rats are oral administrated a dose of 12 mg/kg of DiO and 12 mg/kg of Rhodamine B as an experiment group, and tail intravenous injected the same dose of DiO and Rhodamine B as an I.V. control group. In addition, the rats are intraperitoneally injected cycloheximide 1 hour before oral administration to block lymphatic absorption as a control group. The animals are sacrificed 6 hours after the feeding and 1 hour after the tail intravenous injection. At the time of maximum accumulation, the rats are sacrificed to remove the main organs (heart, lung, liver, spleen, pancreas and kidney) and the distribution of the fluorescent dye is observed by IVIS.

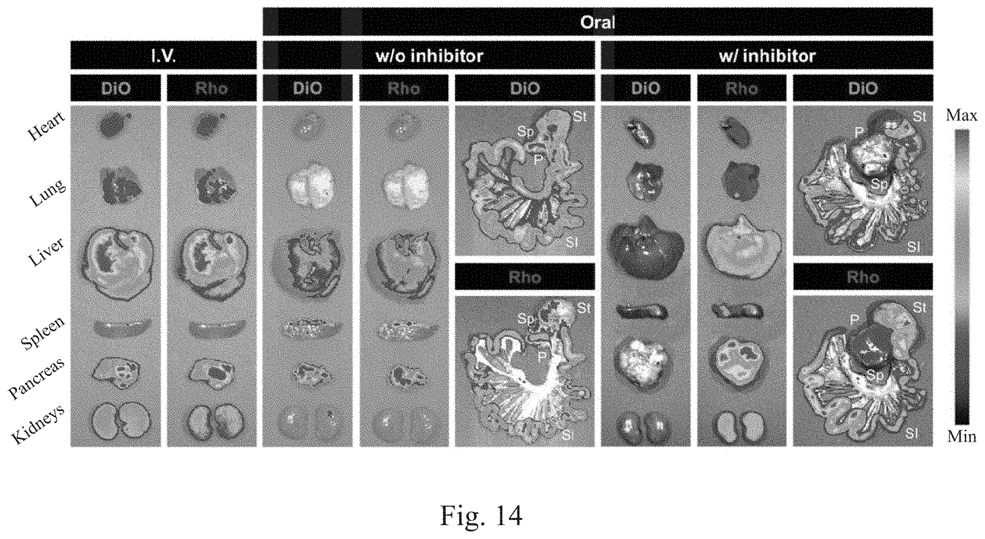

Please refer to FIG. 14, which shows biodistribution of fluorescent model drugs in test rats with different administrations. In FIG. 14, DiO and Rhodamine B are accumulated in liver, pancreas and kidneys in the I.V. control group. In the experiment group and the control group, the Rhodamine B is accumulated in liver, pancreas and the lateral surfaces of intestinal tract. In the experiment group, the DiO is accumulated in liver, pancreas, the lateral surfaces of intestinal tract and mesentery, while the accumulation cannot be detected in the control group. Ex vivo IVIS images demonstrate that the hydrophobic small molecule drug is delivered to the pancreas via intestinal lymphatic transport, and the hydrophilic small molecule drug is delivered to the pancreas via blood circulation.

### 3. Pharmacokinetics of the Pharmaceutical Composition of the Present Disclosure

The pharmacokinetics of the pharmaceutical composition of the present disclosure is analyzed in this experiment. There are two groups, the rats are oral administered of the pharmaceutical composition of the present disclosure with 12 mg/kg of paclitaxel and 12 mg/kg of gemcitabine as the oral group, and the rats are tail intravenous injected at a dose of 12 mg/kg of paclitaxel and 12 mg/kg of gemcitabine as the I.V. group. After administration, blood of rat is collected from the tail vein at 0.5, 1, 2, 3, 4, 6, 8, 10, 12, 24, 48 hours, and plasma of rat is obtained after centrifugation. After treatment, the drug concentration is quantified by HPLC.

For quantifying gemcitabine (hereafter GEM), tetrahydrouridine (10 mM, 10 μL) is added, and 2 mL of an organic mixture (15% isopropanol in ethyl acetate) is added into 100 μL of the plasma. After mixing, the supernatant is centrifuged, dried, and then dissolved in the mobile phase to perform HPLC. Mobile phase is 0.1 M ammonium acetate:acetonitrile=98:2. For quantifying paclitaxel (hereafter PTX), 400 μL of acetonitrile is added into 100μL of plasma. After mixing well, the sample is centrifuged to remove the supernatant. Then 100 μL of ZnSO₄ (10% w/v aqueous solution) is added. After mixing well, the sample is centrifuged to remove the supernatant. Then 1 mL of ethyl acetate is added. After mixing well, the sample is centrifuged to remove the supernatant, and then dried. Then the sample is dissolved in methanol to perform HPLC. Mobile phase is H₂O:acetonitrile=90:10.

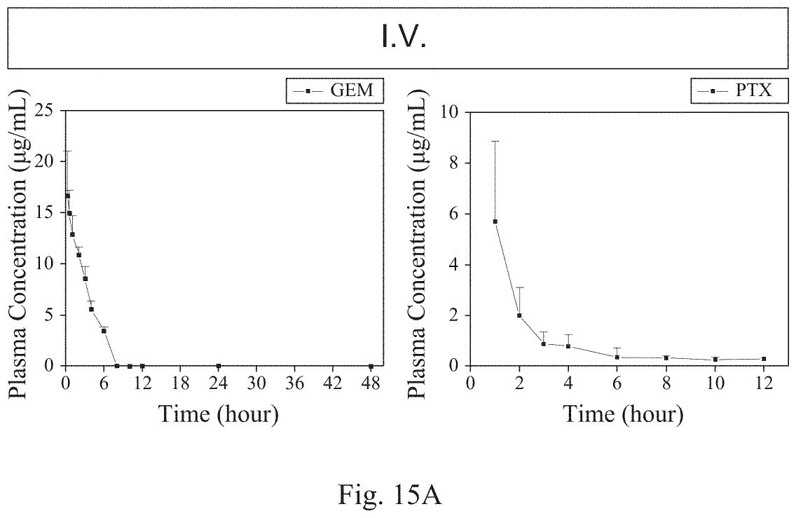

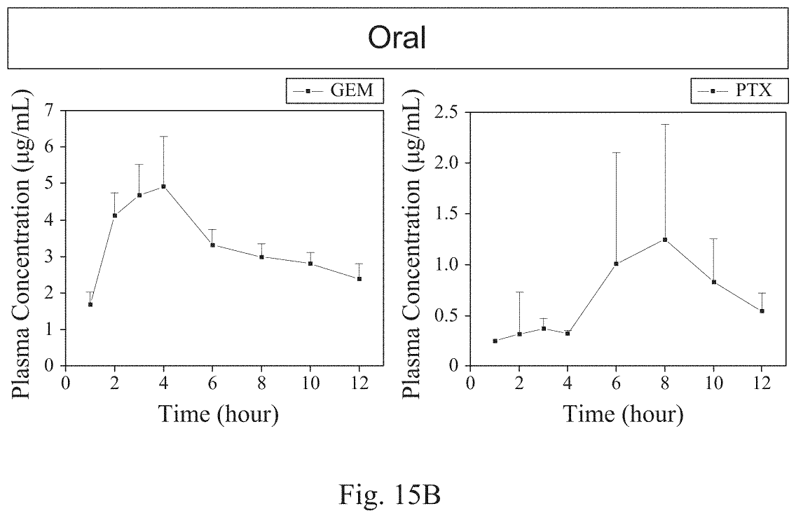

Please refer to Table 1 and FIGS. 15A and 15B, which show the pharmacokinetics of the pharmaceutical composition of the present disclosure, wherein FIG. 15A shows the analysis results in the I.V. group, and FIG. 15B shows the analysis results in the oral group.

TABLE 1

| | | Dose | Cₘₐₓ | T_{maxz} | T_{1/2} | AUC |
|---|---|---|---|---|---|---|
| | | (mg/kg) | (μg/mL) | (h) | (h) | (μg h/mL) |
| I.V. | GEM | 12.0 | 16.66 ± 4.38 | — | 2.36 | 41.13 |
| | PTX | 12.0 | 19.95 ± 5.01 | — | 10.19 | 23.56 |
| Oral | GEM | 12.0 | 3.62 ± 1.38 | 4.00 | 6.13 | 23.38 |
| | PTX | 12.0 | 1.01 ± 0.97 | 8.00 | 14.44 | 7.25 |

| | | AUMC | CL | MRT | F |
|---|---|---|---|---|---|
| | | (μg h²/mL) | (L/h/kg) | (h) | (%) |
| I.V. | GEM | 135.11 | 0.24 | 3.28 | — |
| | PTX | 39.21 | 0.42 | 1.66 | — |
| Oral | GEM | 131.19 | 0.43 | 5.61 | 56.84 |
| | PTX | 52.32 | 1.38 | 7.22 | 59.20 |

In FIG. 15A, the concentration of GEM and PTX are increased rapidly after the administration in the I.V. group, and the concentration of GEM and PTX cannot be detected after 6 hours post-administration. In FIG. 15B, the concentration of GEM reaches the highest peak at 4 hours post-administration, and the concentration of PTX reaches the highest peak at 6 hours post-administration. In addition, the concentration of GEM and PTX still can be detected within 12 hours post-administration. In Table 1, the bioavailability (F) of GEM is 56.84% in the oral group, and the bioavailability of PTX is 59.20% in the oral group. The results indicate that the pharmaceutical composition of the present disclosure has good bioavailability.

### 4. Therapeutic Effect of the Pharmaceutical Composition of the Present Disclosure on Treatment of Cancer

First, the therapeutically effective amount of the hydrophobic small molecule drug and the therapeutically effective amount of the hydrophilic small molecule drug are confirmed in the experiment. An enteric-coated gelatin capsule is filled with powdered hydrophobic small molecule drug, hydrophilic small molecule drug, lipophilic solvent, acidic compound and effervescent ingredient as one example of the pharmaceutical composition of the present disclosure, wherein the hydrophobic small molecule drug is paclitaxel, the hydrophilic small molecule drug is gemcitabine, the lipophilic solvent is capric acid, the acidic compound is citric acid, and the effervescent ingredient is sodium bicarbonate in this experiment. The predetermined dose of paclitaxel is 4, 8 or 12 mg/kg, and the predetermined dose of gemcitabine is 4, 8 or 12 mg/kg.

Tumor rats are established and further used to test the therapeutic effects on the pharmaceutical composition of the present disclosure. On day 0, rats are inoculated with 1×10⁶ DSL-6A/C1 cells mixed with 0.1 mL solution Matergel and medium (1:1 v/v) by 27 G needles orthotopically. An incision is made on the left flank of a rat, and the pancreas is exposed and injected. Treatment is repeated every five days (day 15, 20, and 25). The size of each tumor, which is estimated as length×width×height×π/6, is assessed using a caliper on day 30 after the rat is sacrificed and the tumor is excised. The treatment is that the tumor rats are oral administrated with the pharmaceutical composition of present disclosure with different doses of paclitaxel and gemcitabine.

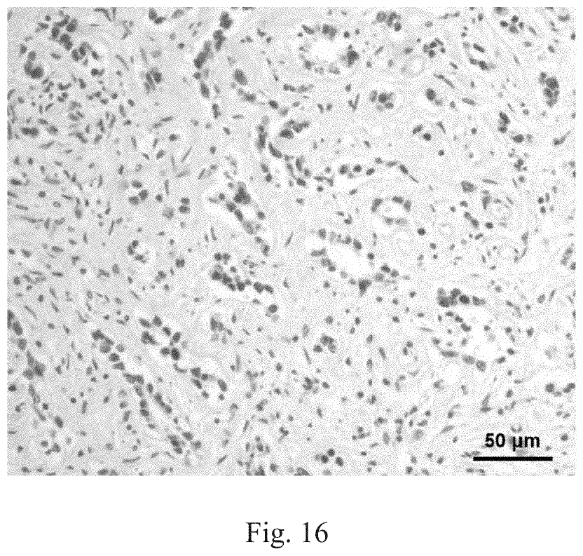

In order to confirm that the tumor rats with pancreatic cancer are established, the pancreatic tumors are excised from the tumor rats and performed hematoxylin and eosin staining. Please refer to FIG. 16, which shows analysis result of the hematoxylin and eosin staining. The result shows that the cell type of the pancreatic tumor is a malignant pancreatic ductal carcinoma.

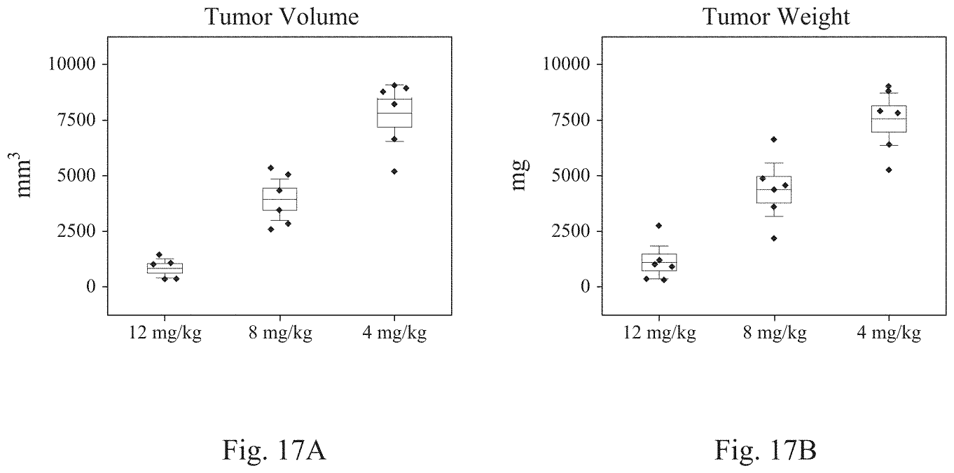

Please refer to FIGS. 17A and 17B, which shows analysis results of the dose dependent study of the pharmaceutical composition of the present disclosure. In FIGS. 17A and 17B, the therapeutic effect of the pharmaceutical composition of the present disclosure on the treatment of cancer is dose-dependent. The results show that the dose of paclitaxel of 12 mg/kg and the dose of gemcitabine of 12 mg/kg have better therapeutic effect. Thus, the dose of paclitaxel of 12 mg/kg and the dose of gemcitabine of 12 mg/kg are selected for further experiments.

Further, the cancer treatment effect on the pharmaceutical composition of the present disclosure is confirmed by treated tumor rats with Example or Comparative Example of the pharmaceutical composition. There are five groups in this experiment, which are the tumor rats oral administrated with three enteric-coated gelatin capsules contained a powdered mixture of 1 mg of paclitaxel, 1 mg of gemcitabine, 18 mg of capric acid, 5 mg of sodium bicarbonate and 6 mg of citric acid in each (represented as “P+G w/F”), the tumor rats oral administrated with three enteric-coated gelatin capsules contained a powdered mixture of 18 mg of capric acid, 1 mg of paclitaxel and 1 mg of gemcitabine in each (represented as “P+G”), the tumor rats oral administrated with three enteric-coated gelatin capsules contained a powdered mixture of 18 mg of capric acid, 5 mg of sodium bicarbonate and 6 mg of citric acid in each (represented as “Empty”), the tumor rats tail intravenous injected with 12 mg/kg of paclitaxel and 12 mg/kg of gemcitabine (represented as “I.V.”), and the tumor rats untreated (represented as “Untreated”). The tumor rats are humanely sacrificed on 30 days post-administration, and the size of each tumor, which is estimated as length×width×height×π/6. The tissue of tumor is cut to size and embedded in OCT gel for frozen section and subsequent staining.

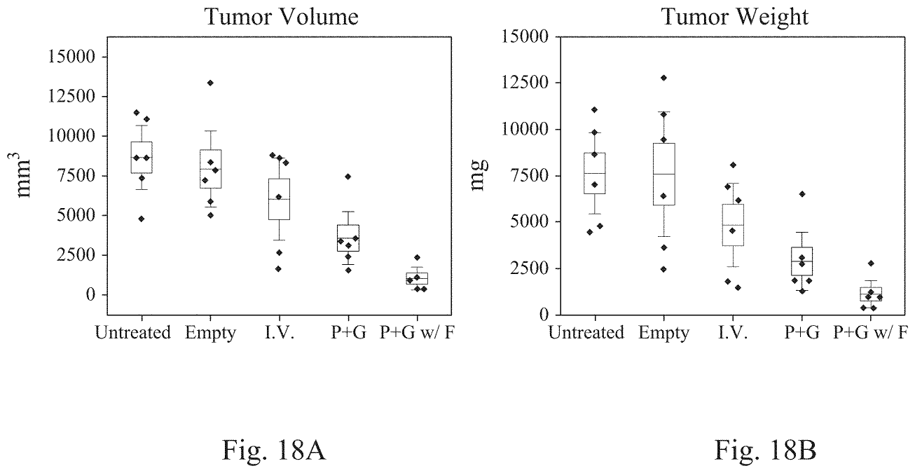

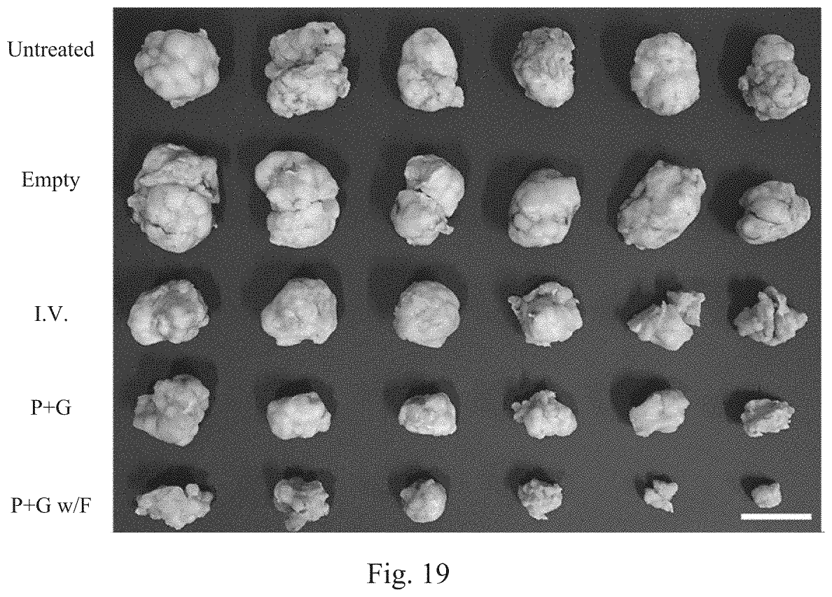

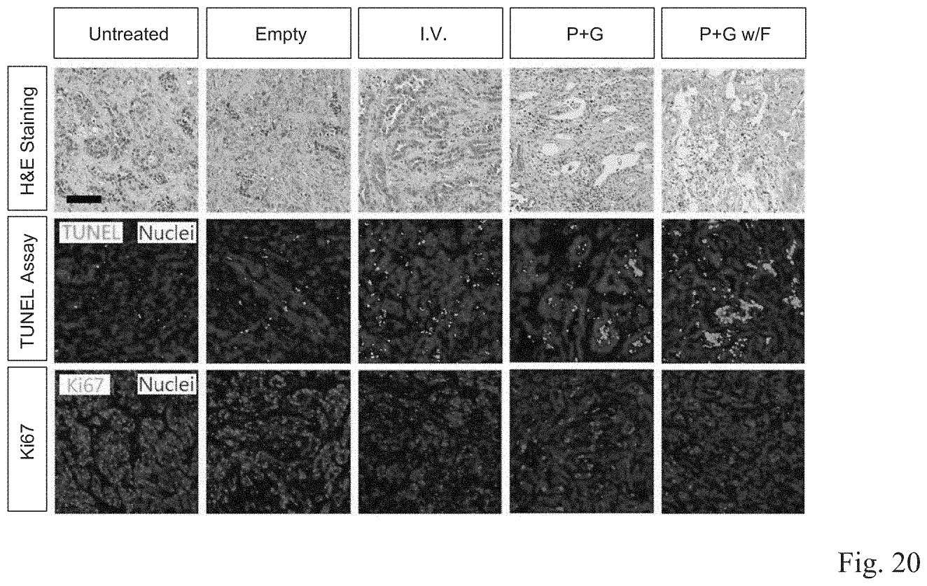

Please refer to FIGS. 18A, 18B, 19 and 20, which show analysis results of the therapeutic efficacy of the pharmaceutical composition of the present disclosure, wherein FIG. 18A shows the tumor volume of the five groups, FIG. 18B shows the tumor weight of the five groups, FIG. 19 shows photo of the tumors of the five groups, and FIG. 20 shows the analysis results of the immunofluorescence staining of the five groups. The results indicate that the growth of the tumor is significantly reduced in the tumor rats treated with the pharmaceutical composition of the present disclosure (P+G w/F) compared with other groups. In addition, analysis results of the immunofluorescence staining indicate that the pharmaceutical composition of the present disclosure (P+G w/F) can reduce the activity of tumor cells.

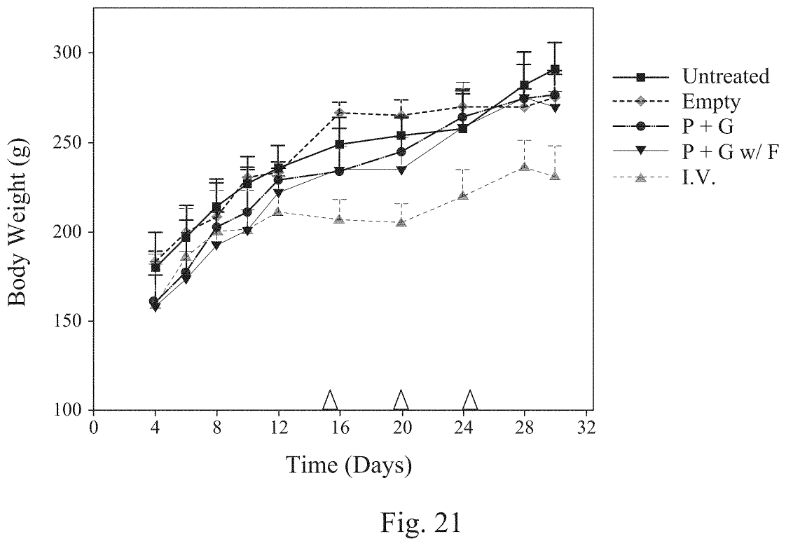

Further, to determine the safety of the pharmaceutical composition of the present disclosure, the body weight of the tumor rats of the five groups are detected on 0, 4, 8, 12, 16, 20, 24, 28 and 32 days post-administration. Please refer to FIG. 21, which shows body weight curve of the tumor rats after different administrations. In FIG. 21, the oral administration of the pharmaceutical composition of the present disclosure (P+G w/F) does not affect the body weight of the tumor rats, indicating that the pharmaceutical composition of the present disclosure is a safe vehicle for delivering the hydrophobic small molecule drug and the hydrophilic small molecule drug.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A pharmaceutical composition for oral delivery of hydrophobic small molecule drug and hydrophilic small molecule drug concurrently, comprising:
an enteric layer; and
a drug layer encapsulated in the enteric layer, comprising:
a therapeutically effective amount of a hydrophobic small molecule drug, wherein a molar mass of the hydrophobic small molecule drug is less than 1000 g/mol;
a therapeutically effective amount of a hydrophilic small molecule drug, wherein a molar mass of the hydrophilic small molecule drug is less than 1000 g/mol;
a lipophilic solvent for dissolving the hydrophobic small molecule drug;
an acidic compound; and
an effervescent ingredient generating carbon dioxide bubbles when the acidic compound is dissolved in intestinal fluid to form an acidic environment;
wherein lipophilic tails of bile salts carry the hydrophobic small molecule drug dissolved in the lipophilic solvent to incorporate into a nanofilm around each of the carbon dioxide bubble to form a monolayer system, then each of the carbon dioxide bubble expands and approaches an air-liquid interface in a lumen, the monolayer system transforms into a double-layer nano-assembly having an inner layer and an outer layer, the hydrophilic small molecule drug is embedded in a gap formed between the inner layer and the outer layer of the double-layer nano-assembly, and lipid oil drops containing the hydrophobic small molecule drug are formed when the carbon dioxide bubbles burst at the air-liquid interface in the lumen.

2. The pharmaceutical composition of claim 1, further comprising a gelatin layer, wherein the drug layer is coated with the gelatin layer.

3. The pharmaceutical composition of claim 2, wherein the pharmaceutical composition is in form of a tablet or a capsule.

4. The pharmaceutical composition of claim 1, wherein the enteric layer comprises a methacrylic acid copolymer, hypromellose phthalate, hydroxypropyl cellulose acetate, hydroxypropyl cellulose succinate, or carboxymethyl ethyl cellulose.

5. The pharmaceutical composition of claim 1, wherein the hydrophobic small molecule drug comprises curcumin, paclitaxel, doxorubicin, cisplatin, mitomycin C, etoposide, irinotecan or tamoxifen.

6. The pharmaceutical composition of claim 1, wherein the hydrophilic small molecule drug comprises gemcitabine or 5-fluorouracil.

7. The pharmaceutical composition of claim 1, wherein the lipophilic solvent is C6-C10 fatty acid.

8. The pharmaceutical composition of claim 7, wherein the C6-C10 fatty acid is capric acid.

9. The pharmaceutical composition of claim 1, wherein the acidic compound is citric acid.

10. The pharmaceutical composition of claim 1, wherein the effervescent ingredient is sodium bicarbonate.

11. The pharmaceutical composition of claim 1, wherein the lipophilic solvent, the acidic compound and the effervescent ingredient of the drug layer are contained in a weight ratio of 18:2:1 to 18:8:7.
